(19) United States
(12) Patent Application Publication
(10) Pub. No.: US 2023/0338128 A1
(43) Pub. Date: Oct. 26, 2023

(54) IMPLANT FOR TREATING VAGINAL PROLAPSE

(71) Applicants: Adrian Paz, Patah Tikva (IL); Yahuda Bachar, Yehuda - Monosson (IL)

(72) Inventors: Adrian Paz, Patah Tikva (IL); Yahuda Bachar, Yehuda - Monosson (IL)

(21) Appl. No.: 17/922,975

(22) PCT Filed: Jun. 14, 2021
(86) PCT No.: PCT/IL2021/050714

§ 371 Date: Nov. 3, 2022

Related U.S. Application Data
(60) Provisional application No. 63/041,188, filed on Jun. 19, 2020.

(51) Int. Cl. A61F 2/844; A61F 2/00

(52) U.S. Cl. CPC A61F 2/005; A61F 2/844; A61F 6/12

(57) ABSTRACT

Some embodiments an implant supports female genitlia, for example, to treat and/or prevent prolapse and/or urinary incontinence. The implant may be inserted into a vagina non-surgically. Optionally, the device may be incorporated within the vaginal wall. Optionally, the device includes a hollow elastic frame. For example, the frame may be made of metal and/or wire and/or a cut out and/or a polymer. In some embodiment a cranial portion of the device is designed for supporting the vaginal dome. For example, the cranial end of the device may include an element (e.g., support wires) running in an anterior-posterior direction. Optionally there may be a free space avoiding interference with the uterine cervix. In some embodiments, a caudal portion of the device is configured to hold the device in the vagina. For example, a caudal end of the device may be configured to rest on the pelvic floor

### RELATED APPLICATION/S

This application claims the benefit of priority under 35 USC §119(e) of U.S. Provisional Pat. Application No. 63041188 filed 19 Jun. 2020, the contents of which are incorporated herein by reference in their entirety.

### FIELD AND BACKGROUND OF THE INVENTION

The present invention, in some embodiments thereof, relates to a device for treatment for vaginal prolapse and, more particularly, but not exclusively, to a device and method for reduced invasive treatment of genital prolapse in females.

U.S. Pat. Application Publication No. 20090112052 appears to disclose, “an implant for placement in the retropubic space of a patient. Novel methods and assemblies for use in conjunction with the implant are also described, which include mechanical positioning of the sling, placement of a mechanical implant underneath the urethra or mechanical vibration (intermittent) under the urethra or other incontinence lumen.”

U.S. Pat. No. 6413206 appears to disclose, “An intra-vaginal device (30) to aid in controlling urinary incontinence. The device (30) has a base (31) from which there projects a rear part (34). The rear part (34) engages the posterior vaginal wall and rests on the pelvic floor and projects towards the cervix. The base (31) has a convex surface (33) which engages the anterior vaginal wall to support the vaginal wall and the urethra therebehind.”

U.S. Pat. No. 10188345 appears to disclose, “a flexible and non-absorbent vaginal insert device shaped to be held securely in place in a vagina when inserted, so as to improve symptoms associated with pelvic organ prolapse or urinary incontinence or both, is provided. The device is designed to be relatively easy to insert and remove. An applicator may be used to aid with insertion. The vaginal insert device comprises an upper portion, having a cone-shaped body, having a circular transverse cross-section throughout its length, having a wall with an interior side and an exterior side, an upper open end, a lower end, and a hollow interior, wherein the upper open end of the upper portion is the innermost portion of the device during insertion, and wherein the wall of the upper portion can be squeezed to make the upper portion more compact for easier insertion of the device, and wherein after insertion the wall expands back to its original shape. The device further comprises an exterior rim surrounding and protruding from the exterior side of the wall and being adjacent to the upper open end, and a plurality of ridges surrounding and protruding from the exterior side of the wall, and a removal portion extending from said lower end of said upper portion to assist in removal of the device. The device can also comprise one or more ventilation openings.”

U.S. Pat. No. 8337391 appears to disclose, “an implant comprising a support body from which there extends at least to upper suspension stabilizers each disposed on either side of a sagittal plane on a longitudinal axis, the longitudinal axes forming between them an angle greater than 45°, and two lower suspension stabilizers disposed on either side of the sagittal plane; and an introduction device for inserting the implant in the woman, the introduction device shaped analogous to that of the implant and comprising a hollow body defining a cavity configured to receive the support body of the implant, tubular straps extending from the hollow body with each defining a cavity for receiving one of the suspension stabilizers of the implant, traction means extending from an end of each of the tubular straps, and a cut initiator provided on the hollow body.” a medical device configured to be delivered and placed within a patient’s body. The medical device includes an implant and a cover member. The implant is configured to be disposed within the patient’s body. The implant includes a support member, a first arm member and a second arm member, an elongated first sleeve, and an elongated second sleeve. The support member includes a crescent shaped opening.”

U.S. Pat. 9814555 appears to disclose, “a medical device configured to be delivered and placed within a patient’s body. The medical device includes an implant and a cover member. The implant is configured to be disposed within the patient’s body. The implant includes a support member, a first arm member and a second arm member, an elongated first sleeve, and an elongated second sleeve. The support member includes a crescent shaped opening.”

U.S. Pat. No. 10045842 appears to disclose, “embodiments of surgical procedure systems, devices, tools, and methods, useful for treating pelvic conditions in a male or female, the pelvic conditions including incontinence (various forms such as fecal incontinence, stress urinary incontinence, urge incontinence, mixed incontinence, etc.), vaginal prolapse (including various forms such as enterocele, cystocele, rectocele, apical or vault prolapse, uterine descent, etc.), and other conditions caused by muscle and ligament weakness, the devices and tools including devices and tools for anchoring an implant to tissue, devices and tools for transvaginally accessing a posterior region of pelvic anatomy, devices (including certain types of implants, anchors, and tools) for connecting (e.g., adjustably) a vaginal apex to a region of sacral anatomy to provide support to the vaginal apex, and related methods.”

Additional background art includes U.S. Pat. No. 8801593, 20150018941, U.S. Pat.Application Publication No. 20130296643, and U.S. Pat. Application Publication No. 10470861.

### SUMMARY OF THE INVENTION

According to an aspect of some embodiments of the invention, there is provided an intravaginal device including: a hollow elastic filament frame configured for intimate contact with vaginal walls; a cranial end configured for supporting a vaginal dome and a caudal end configured for resting on a pelvic floor for preventing dislodgment.

According to some embodiments of the invention, the cranial end include members running in an anterior-posterior direction positioned to contact the vaginal dome on opposite lateral sides of a cervix.

According to some embodiments of the invention, the caudal end includes one or more lateral running elements configured for resting on the pelvic floor and inhibiting dislodgment of the device.

According to some embodiments of the invention, the filament has a diameter between ⅒ to 5 mm.

According to some embodiments of the invention, the filament has a diameter between 3/10 to 3/2 mm.

According to some embodiments of the invention, the caudal end is configured to rest on a perineal body.

According to some embodiments of the invention, the caudal end is configured to rest on a pubic symphysis.

According to some embodiments of the invention, the caudal end configured to rest on the pubic symphysis is provided with a notch to prevent pressure on a urethra.

According to some embodiments of the invention, the frame delineates an axial passage for permitting unhindered sexual intercourse.

According to some embodiments of the invention, the filament is made of a super-elastic material.

According to some embodiments of the invention, the super-elastic material is selected from at least one of Nitinol and an elastic polymer.

According to some embodiments of the invention, the frame is made of a nitinol wire.

According to some embodiments of the invention, the frame is cut from a sheet.

According to some embodiments of the invention, the frame is formed from a bent ring.

According to some embodiments of the invention, the frame is saddle shaped with two caudal extensions.

According to some embodiments of the invention, frame has a figure of eight shape including two loops each loop having a crossing point and two arched extensions connecting between the two crossing points.

According to some embodiments of the invention, cranial end forms an arc.

According to some embodiments of the invention, the cranial end of the device includes two lateral wires, that are configured to support on opposite lateral sides of a uterine cervix.

According to some embodiments of the invention, the frame is coated with a biocompatible material chosen from a group of: PTFE, POLYURETHANE, SILICONE.

According to an aspect of some cmbodiments of the invention, there is provided a method for treating vaginal prolapse including: inserting an open frame into a vagina; placing the frame into intimate contact with vaginal walls and mucosa supporting a vaginal vault with a cranial end, of the open frame, and resting an caudal end of the frame on a pelvic floor.

According to some embodiments of the invention, the method further includes: resting posterior caudal end of the frame on a perineal body.

According to some embodiments of the invention, the method further includes: incorporating the frame into vaginal walls.

According to some embodiments of the invention, the method further includes: compressing the frame before the inserting and expanding the frame before the placing.

According to some embodiments of the invention, the method further includes introducing the frame into a delivery tube prior to the inserting.

According to some embodiments of the invention, the placing is done gradually.

According to some embodiments of the invention, the placing is controlled using a collapsing element.

According to some embodiments of the invention, the collapsing element includes a filament wrapped around the frame.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a perspective view of a vaginal stent in accordance with an embodiment of the current invention;

FIG. 2 is a is a schematic view of a stent installed in a vagina in accordance with an embodiment of the current invention;

FIG. 3 is a is a block diagram of a vaginal support device in accordance with an embodiment of the current invention;

FIG. 4 is a block diagram of a vaginal support device in accordance with an embodiment of the current invention;

FIG. 5 is a is a flowchart illustration of a method of a vaginal stent in accordance with an embodiment of the current invention;

FIG. 6 is a schematic cross sectional view of a stent in a delivery device in accordance with an embodiment of the current invention;

FIG. 7 is a block diagram of a delivery device in accordance with an embodiment of the current invention;

FIG. 8 is a flowchart illustration of a method of delivering a vaginal stent in accordance with an embodiment of the current invention;

FIG. 8 is a perspective view of a bimodal bent wire stent in accordance with an embodiment of the current invention;

FIG. 9 is a perspective view of a bent figure eight wire stent in accordance with an embodiment of the current invention;

FIG. 10 is a perspective view of an undulating ring single filament stent in accordance with an embodiment of the current invention;

FIG. 11 is a perspective view of an undulating ring single filament stent in accordance with an embodiment of the current invention ;

FIG. 12 is a schematic illustration of a stent being prepared for deployment in accordance with an embodiment of the current invention;

FIG. 13 illustrated a collapsed stent positioned inside a delivery tube in accordance with an embodiment of the current invention;

FIG. 14 is a schematic illustration of a stent in a partially deployed state in accordance with an embodiment of the current invention;

FIG. 15 is a is a schematic frontal view of a device including an installed in a vagina in accordance with an embodiment of the current invention; and

FIG. 16 is a is a schematic perspective view of female internal genitalia and a device including installed in a vagina in accordance with an embodiment of the current invention.

### DESCRIPTION OF SPECIFIC EMBODIMENTS OF THE INVENTION

The present invention, in some embodiments thereof, relates to a device for treatment for vaginal prolapse and, more particularly, but not exclusively, to a device and method for reduced invasive treatment of genital prolapse in females.

### Overview

An aspect of some embodiments of the current invention relates to a device (e.g., an implant and/or a stent) configured to support female genitlia. For example, the device may be used to treat and/or prevent prolapse and/or stress urinary incontinence. In some embodiment the device may be configured for insertion into a vagina, for example, non-surgically. Optionally, the device may be configured to be incorporated within the vaginal wall. For example, over time a portion and/or all of the device may be covered by the mucosa (e.g., thin wires of the frame may be incorporated within the vaginal wall with time being covered by the mucosa). Optionally, the device may be configured for permanent residency in the user. Alternatively or additionally, the device may remain in place for long and/or short term and/or be removed and/or be self removed (e.g., part or all of the device may dissolve and/or be excreted after a period of time). Optionally, the device includes a hollow frame. Optionally, the frame is elastic. For example, the frame may be made of metal and/or wire and/or a cut out and/or a polymer. Optionally, the device is configured to be in intimate contact with the vaginal walls. Optionally when the stent expands in to the vagina, some or all of the filaments of the stent will become invaginated into the tissue (e.g., mucus tissue) of the walls of the vagina. Optionally, over the time vaginal tissue will grow over the filaments and/or incorporate the stent into the vaginal tissue. Optionally, the perimeter of the device may be open. For example, the perimeter of the device may include filaments over less than 1% of its surface area and/or less than 5% and/or less than 10% and/or less than 0.3% and/or less than 0.1%.

In some embodiment a cranial portion of the device is designed for supporting the vaginal dome (e.g., the fornix). For example, the cranial end of the device may include an element (e.g., support wires) running in an anterior-posterior direction. Optionally there may be a free space and/or a free path along the anterior and/or cranial sides thereof. For example, this free path may avoid interference with the uterine cervix. For example, the device may be configured to allow a user to become pregnant. For example, lateral support members may be disconnected in a cranial region of the device (e.g., the cranial ½ and/or ¼ and/or ⅛ and/or 1/16 of the device). Optionally, the cranial portion may be arched for stability.

In some embodiments, a caudal portion of the device is configured to hold the device in the vagina. For example, a caudal end of the device may be configured to rest on the pelvic floor e.g., for inhibiting dislodgment. For example, a caudal anterior portion and/or end of the device may include an element that is configured to rest on Pubocervical fascia and/or the pubic symphysis. Optionally, the caudal anterior element may include a lateral running element (e.g., a wire) and/or a feature (e.g., a bend and/or a notch) to avoid interfering with the urethra. For example, a caudal posterior portion and/or end of the device may include an element that is configured to rest on Rectovaginal fascia and/or a perineal body. Optionally, the caudal posterior element may include a lateral running element (e.g., a wire). Optionally, the device includes an axial passage that facilitates sexual intercourse while the device is in place.

In some embodiments, the device may have the form of a frame made of wire, polymer and/or other materials. For example, wires may have a width/diameter ranging between 0.1 to 5 mm and/or between 0.3 to 1.5 mm. The device may have a width (e.g., in the lateral and/or anterior/posterior direction) for example ranging between 10 to 30 mm and/or between 30 to 100 mm and/or between 100 to 200 mm. The device may have a length (e.g., in the cranial/caudal direction) for example ranging between 20 to 40 mm and/or between 40 to 150 mm and/or between 150 to 300 mm and/or between 300 to 500 mm. Optionally, the frame may be made of a super-elastic material such as: supcr-clastic metallic alloy such as but not limited to: Nitinol and/or an elastic polymer. Optionally, the frame is made of wire. Alternatively or additionally, the frame may be cut from a sheet. Optionally, the frame is coated and/or covered with a biocompatible material (e.g., PTFE, Polyurethane, Silicone).

In some embodiments, a stent is formed of an open frame. For example, the cranial portion of the frame (e.g., the cranial ½ and/or ¼ and ⅛) may not include a full ring. For example, this may inhibit strangulation of the cervix (for example, even if the cervix were to prolapse into the cranial portion of the stent, it would not be surrounded on all sides, avoiding strangulation. For example, the cranial portion of the stent may include disconnected lateral retractors. The anterior/posterior running connectors between the retractors are optionally only caudal to the cranial portion of the stent. More generally, the cranial portion of the stent may include disconnected retractors (lateral, anterior, posterior or otherwise). The connectors between the retractors are optionally only caudal to the cranial portion of the stent. In some embodiments, the frame may take a form that is not spiral.

### Exemplary Embodiments

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

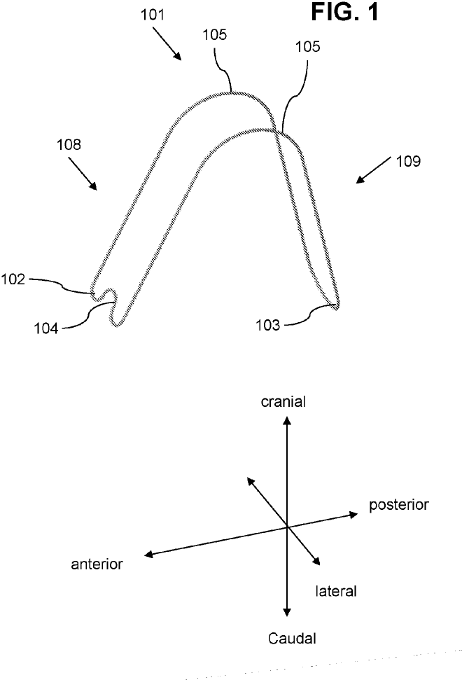

FIG. 1 is a perspective view of a vaginal stent in accordance with an embodiment of the current invention. Optionally, the stent is formed of thin flexible members and/or has a mostly open structure that is configured to incorporate into tissue (e.g., a mucus membrane and/or muscle of the vaginal wall). Optionally, the device includes a ring of one or more filament (for example wire). For example, the ring may be bent into a saddle shape. Optionally, the middle of the saddle is a cranial arch 101 and/or includes a pair of laterally spaced anterior/posterior running cranial arches 105. For example, the arches 105 may be configured for supporting a vaginal dome. For example, the ring may be bent to form two laterally spaced arches 105 spanning an anterior/posterior space (e.g., the opening of the arch may be substantially parallel to the sagittal plane. Optionally, the arches are joined at a caudal portion (e.g., caudal end) thereof (e.g., forming two caudal extensions e.g., an anterior extension 108 and/or a posterior extension 109). For example, each caudal extension 108, 109 may include two laterally separated elements (e.g., wires) jointed at a caudal end by a lateral running element 102, 103 (e.g., filament and/or wire) may join the one or both of the caudal ends of the laterally separated elements. Optionally, a caudal anterior element may include a notch 104, for example, in the middle thereof, for example to make space for and/or avoid interfering with a urethra. For example, the laterally separated element of the arches 105 and caudal extensions 108, 109 may retract the walls and/or dome of the vagina. Optionally, the space between the laterally spaced elements of the arches 105 and/or the anterior extension 108 are configured such that the opening of the cervix fits between the laterally separated clements. For example, there may not be lateral connecting elements on the cranial portion of the device. In some embodiments, this will inhibit strangulation of the cervix. Optionally, elements of the stent may be bent and/or undulating. For example, the wires of the retractors (e.g., extensions 108, 109 and/or arches 105 may undulate and/or be bent inward to provide more surface for support and/or incorporation into vaginal tissue). Optionally, anchors (for example a caudal portion of extensions 108, 109) may be bent outwards to make better contact with a supporting feature.

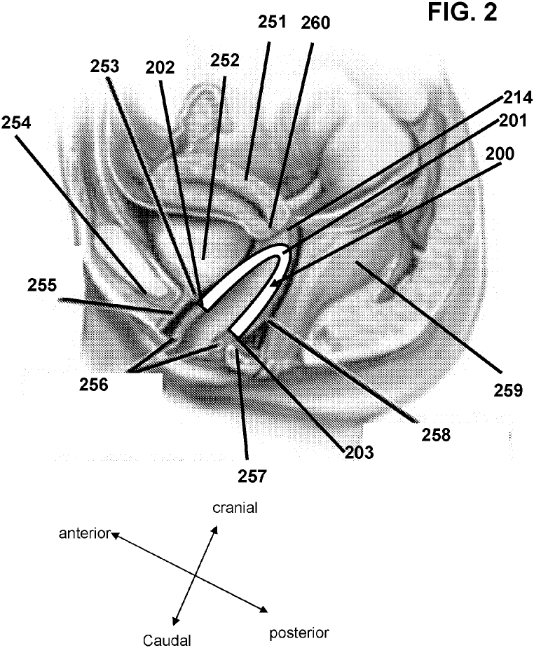

FIG. 2 is a is a schematic view of a device including a stent 200 installed in a vagina in accordance with an embodiment of the current invention. Various anatomical features are illustrated for example, a uterus 251, a bladder 252, Pubocervical fascia 253, a pubic symphysis 254, a urethra 255, Vaginal epithelium 256, a perineal body 257, Rectovaginal fascia 258, a rectum 259, fallopian tubes 1663 ovaries 1664 and a cervix 260. In some embodiments, the device 200 includes a hollow elastic frame. Optionally, the frame is inserted into the vagina, in proper orientation with the cranial end 201. For example, the cranial end 201 may include an arch and/or support a vaginal vault 214. In some embodiments, an anterior caudal end 202 of the device rests on the Pubocervical fascia 253 and/or a pubic symphysis 254. Additionally or alternatively, a posterior caudal end 203 of the device may rest on the perineal body 257 and/or rectovaginal fascia 258. and the wire frame being in intimate contact with the vaginal walls and mucosa.

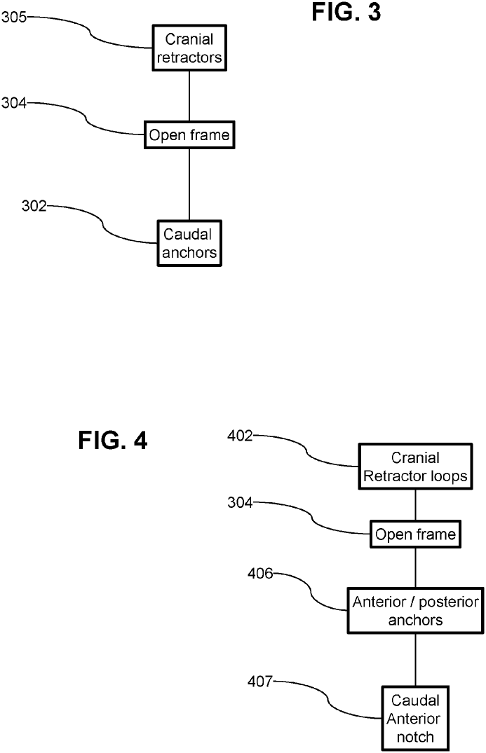

FIG. 3 is a is a block diagram of a vaginal support device in accordance with an cmbodiment of the current invention. In some cmbodiments the device may include a stent. Optionally, the device includes lateral retractors 305. For example, the lateral retractors 305 may include a support for a vaginal vault and/or or vaginal walls. Optionally, a cranial and/or anterior portion of the retractors 305 is open in the middle, e.g., to make space for a cervix and/or to inhibit strangulation of the cervix. Optionally, the retractors include an open frame 304 connecting between the cranial and caudal portions of the device. For example, the lateral retractors may be connected across the frame 304 and/or at a caudal portion of the device. For example, the connection may include one or more caudal anchors 302. For example, the anchors may connect between the lateral retractors and/or rest on a caudal portion of the vagina (e.g., a Pubocervical fascia and/or a pubic symphysis and/or a perineal body and/or rectovaginal fascia). For example, the stent may be anchored to geometrical structures (e.g., near the opening of the vagina).

FIG. 4 is a block diagram of a vaginal stent in accordance with an embodiment of the current invention. In some embodiments the device may include a stent. Optionally, the device includes lateral retractors. For example, the lateral retractors may include a support for a vaginal vault and/or or vaginal walls. Optionally, a cranial and/or anterior portion of the retractors is in the form of an open loop 402, e.g., to make space for a cervix and/or to inhibit strangulation of the cervix. Optionally, the retractors include an open frame 304 connecting between the cranial and caudal portions of the device. For example, the lateral retractors may be connected across the frame 304 and/or at a caudal portion of the device. For example, the connection may include one or more caudal anchors 406, 407. For example, the anchors 406, 407 may connect between the lateral retractors and/or rest on a caudal portion of the vagina (e.g., a Pubocervical fascia and/or a pubic symphysis and/or a perineal body and/or rectovaginal fascia). Optionally, the anterior anchor 406 is configured to avoid interfering with the urethra. For example, the anchor 406 may include a notch and/or a bend at the location of the urethra.

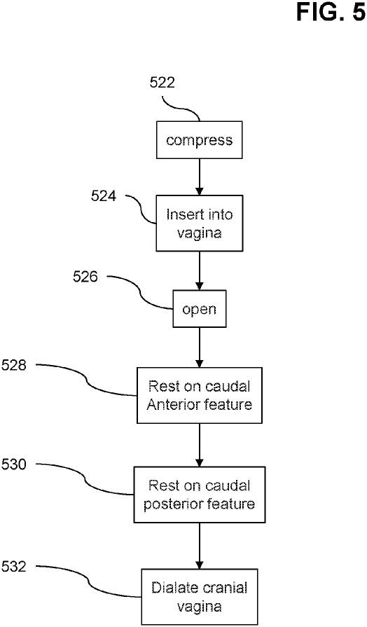

FIG. 5 is a is a flowchart illustration of a method of a vaginal stent in accordance with an embodiment of the current invention. In some embodiments, the stent is compressed 522 before use. Optionally, the compressed state, the device is inserted 524 into the vagina of a patent. For example, the device may be put into a deliver device (for example, a hollow tube) which may be inserted into the vagina and/or the device may be extracted out of the delivery device within the vagina. In the vagina, the device is optionally opened 526 (e.g., expanded). For example, an anchor of the device may rest on a geometric feature of the vagina (for example, an anterior caudal anchor of the device may rest 528 on the Pubocervical fascia and/or a pubic symphysis and/or a posterior caudal anchor of the device may rest 530 on the perineal body and/or rectovaginal fascia. Optionally a cranial portion of the device expands to support and/or dilate 532 the walls of the vagina and/or a prolapsed organ and/or the vaginal dome. Optionally, over time some or all of the stent will be incorporated into vaginal tissue. For example, the stent may be made of filaments (such as biocompatible wire) that will be enveloped into mucus tissue on the walls of the vagina.

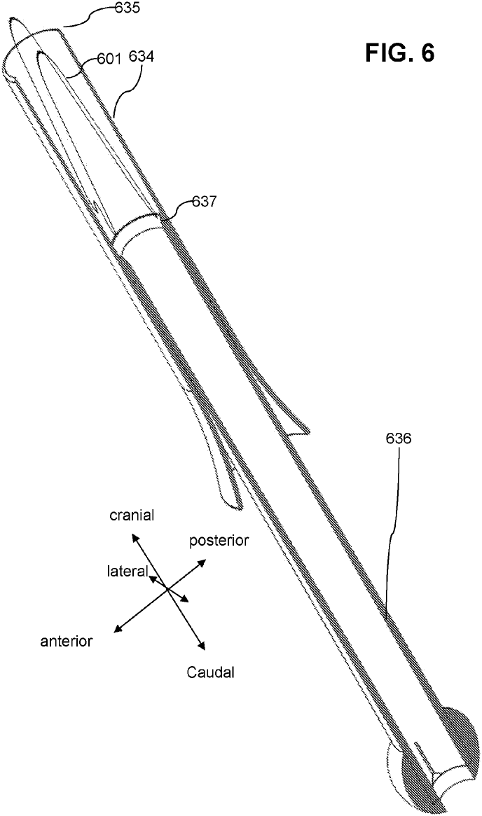

FIG. 6 is a schematic cross sectional view of a stent in a delivery device in accordance with an embodiment of the current invention. In some embodiments a delivery device includes a hollow tube 634 configured to fit the implant 601 (for example, implant 601 may include any of the stents and/or implants and/or devices disclosed herein. Optionally, the implant 601 is introduced into the tube 634. Optionally, the implant is collapsed before being introduced into the tube 634. The distal end of tube (e.g., the cranial end as illustrated in FIG. 6) is pushed into the vagina. Once the distal (cranial) end 635 of tube 634 has reached the area that needs to be supported (e.g., the vaginal dome) and/or once the proximal (caudal) end 637 of device 601 has been inserted until the anchoring point where it will rest, tube 634 is retracted caudally from the vagina while pusher 636 is held steady (for example, tube 634 slides in the proximal direction along the pusher 636). Optionally, pusher 636 holds implant 601 steady as tube 634 is retracted. Once tube 634 has been retracted away from the device 601, device 601 optionally expands to support such that the cranial end of the device 601 supports the cranial end or the vagina and/or to the caudal end 637 of the device contacts and/or anchors itself to features on the caudal portion of the vagina. The tube may have a width (e.g., in the lateral and/or anterior/posterior direction) for example ranging between 10 to 30 mm and/or between 30 to 100 mm and/or between 100 to 200 mm. The tube may have a length (e.g., in the cranial/caudal direction) for example ranging between 20 to 40 mm and/or between 40 to 150 mm and/or between 150 to 300 mm and/or between 300 to 500 mm. An opening in the tube may fit the implant in a compressed state. For example, the opening may have a diameter ranging between 2 to 6 mm and/or between 6 to 20 mm and/or between 20 to 40 mm and/or between 40 to 100 mm. Additionally or alternatively, the delivery device may include a pusher configured to push the stent out of the tube. For example, the pusher may have a length close to the length of the tube (and/or slightly longer) and/or a distal portion of the pusher may fit into the opening of the tube and/or slide along the inside of the tube. Optionally, the

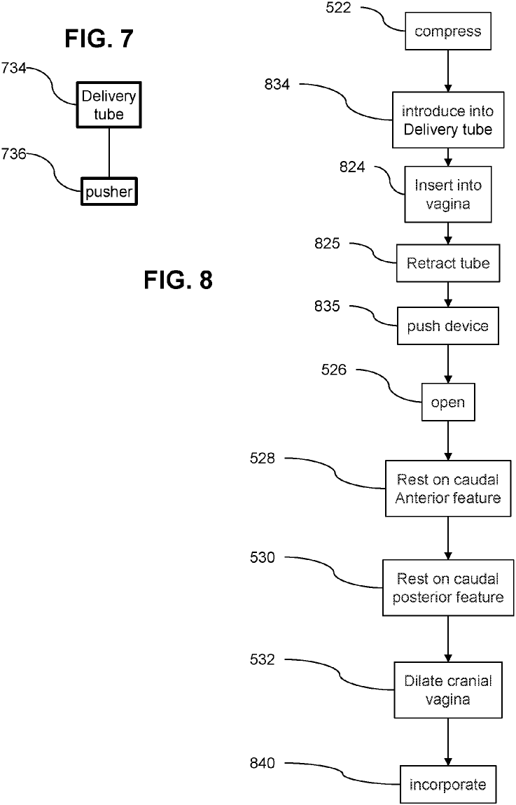

FIG. 7 is a block diagram of a delivery device in accordance with an embodiment of the current invention. For example, the device may include a delivery tube 734 configured to fit into the vagina of a patient. The tube 734 may have a width (e.g., in the lateral and/or anterior/posterior direction) for example ranging between 10 to 30 mm and/or between 30 to 100 mm and/or between 100 to 200 mm. The tube 734 may have a length (c.g., in the cranial/caudal direction) for example ranging between 20 to 40 mm and/or between 40 to 150 mm and/or between 150 to 300 mm and/or between 300 to 500 mm. An opening in the tube 734 may fit the implant in a compressed state. For example, the opening may have a diameter ranging between 2 to 6 mm and/or between 6 to 20 mm and/or between 20 to 40 mm and/or between 40 to 100 mm. Additionally or alternatively, the delivery device may include a pusher 736 configured to push the stent out of the tube 734. For example, the pusher 736 may have a length close to the length of the tube 734 (and/or slightly longer) and/or a distal portion of the pusher 736 may fit into the opening of the tube 734 and/or slide along the inside of the tube 734. Optionally, the tube 734 and/or the pusher 736 may be made of a bio-compatible material (such as stainless steel and/or a polymer). Optionally, the tube 734 and/or the pusher 736 is designed for single use and/or for disposal in municipal garbage. Alternatively or additionally, the tube 734 and/or the pusher 736 are designed for easy sterilization.

FIG. 8 is a flowchart illustration of a method of delivering a vaginal stent in accordance with an embodiment of the current invention. Optionally, the compression 522, the stent may be introduced 834 into a delivery tube. The tube and stent are optionally introduced 824 into the vagina. The device is optionally retained in the vagina by pushing 835 it with a pusher as the tube is retracted 825 and/or removed, allowing the device to open 526 and/or contact the walls of the vagina. Optionally, over time the device is incorporated 840 into the vagina and/or remains permanently and/or semi-permanently (for example, for a period of from 1 day to 1 month and/or from one month to one year and/or from 1 year to six years and/or from six years or more).

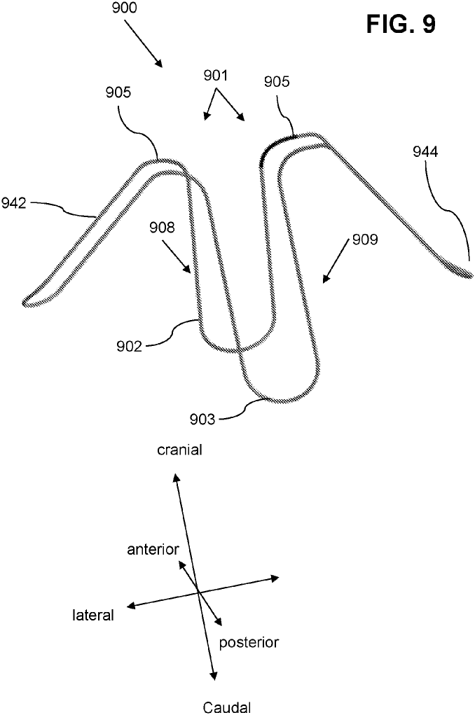

FIG. 9 is a perspective view of a bimodal bent wire stent 900 in accordance with an embodiment of the current invention. Optionally, the stent 900 is formed of thin flexible members and/or has a mostly open structure that is configured to incorporate into tissue (e.g., a mucus membrane and/or muscle of the vaginal wall). Optionally, the device includes a ring of one or more filament (for example wire). For example, the ring may be bent into a bimodal saddle shape. Optionally, the middle of the saddle includes dual pairs 901 of cranial arches 905 and/or includes two pairs 901 of laterally spaced anterior/posterior running cranial arches 905. For example, the arches 905 may be configured for supporting a vaginal dome. For example, the ring may be bent to form two anterior/posterior spaced pairs 901 of laterally spaced arches 905 spanning an anterior/posterior space (e.g., the opening of the arch may be substantially parallel to the sagittal plane. Optionally, the arches 905 of each pair 901 are joined at a caudal portion (e.g., caudal end) thereof (e.g., forming two caudal extensions e.g., an anterior extension 908 and/or a posterior extension 909). In some embodiments, each pair 901 of arches 905 is bent outward (e.g., laterally) to form extended lateral retractors 942. Optionally, the end 944 (e.g., the caudal end) of the retractors is bent outwards (e.g., this may provide improved anchoring of the stent 900 in the walls of the vagina). For example, each caudal extension 908, 909 may include two laterally separated elements (e.g., wires) jointed at a caudal end by a lateral running element 902, 903 (e.g., filament and/or wire) may join the one or both of the caudal ends of the laterally separated elements. Optionally, a caudal anterior element may include a notch, for example, in the middle thereof, for example to make space for and/or avoid interfering with a urethra. For example, the laterally separated element of the arches 905 and caudal extensions 908, 909 may retract the walls and/or dome of the vagina. Optionally, the space between the laterally spaced elements of the arches 905 and/or the anterior extension 908 are configured such that the opening of the cervix fits between the laterally separated elements. For example, there may not be lateral connecting elements on the cranial portion of the device. In some embodiments, this will inhibit strangulation of the cervix. Optionally, elements of the stent 900 may be bent and/or undulating. For example, the wires of the retractors (e.g., extensions 908, 909 and/or arches 905 may undulate and/or be bent inward to provide more surface for support and/or incorporation into vaginal tissue). Optionally, anchors (for example a caudal portion of extensions 908, 909) may be bent outwards to make better contact with a supporting feature.

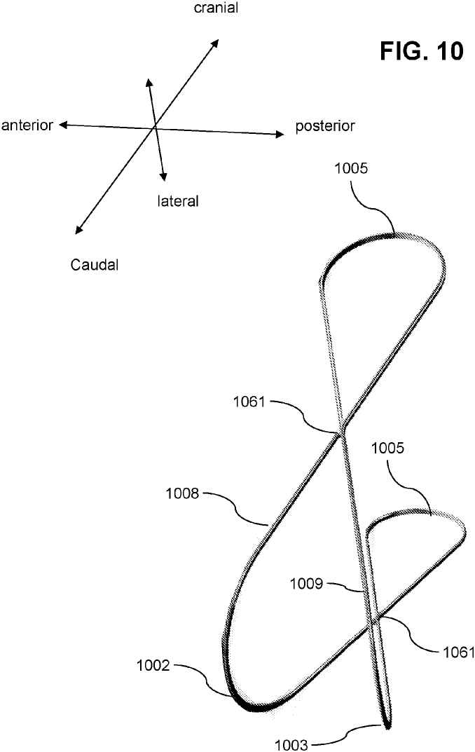

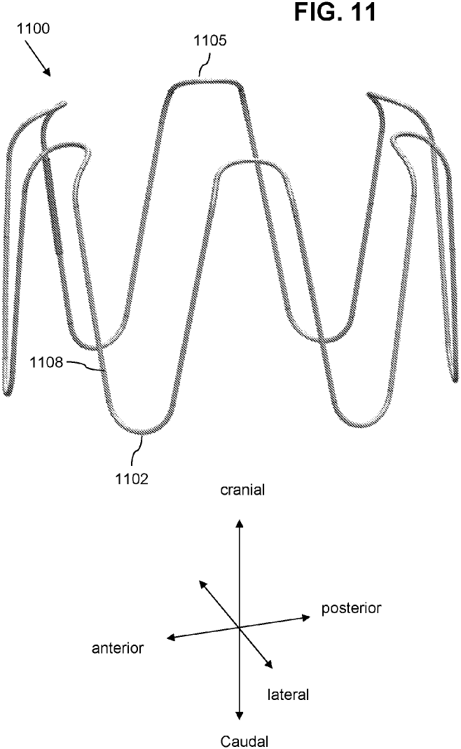

FIG. 10 is a perspective view of a bent figure eight wire stent in accordance with an embodiment of the current invention. Optionally, the stent is formed of thin flexible members and/or has a mostly open structure that is configured to incorporate into tissue (e.g., a mucus membrane and/or muscle of the vaginal wall). Optionally, the device includes a ring of one or more filament (for example wire). For example, the ring may be bent into a 3D FIG. 8 shape. For example, two extension 1009 and 1003 joint between the two loops at crossing points 1061 and 1062. Optionally, cranial end of the stent includes a pair of laterally spaced anterior/posterior running loops 1005. For example, the loops 1005 may be configured for supporting a vaginal dome. For example, the ring may be bent such that the wires cross to form two laterally spaced loops 1005 spanning an anterior/posterior space (e.g., the loop 1005 may be substantially parallel to the sagittal plane. Optionally, the loops 1005 are joined at a caudal portion (e.g., caudal end) of the device (e.g., forming two caudal extensions e.g., an anterior extension 1008 and/or a posterior extension 1009). For example, each caudal extension 1008, 1009 may include two laterally separated elements (e.g., wires) jointed at a caudal end by a lateral running element 1002, 1003 (e.g., filament and/or wire) may join the one or both of the caudal ends of the laterally separated elements. Optionally, a caudal anterior element 1002 may include a notch, for example, in the middle thereof, for example to make space for and/or avoid interfering with a urethra. For example, the laterally separated loops 1005 and caudal extensions 1008, 1009 may retract the walls and/or dome of the vagina. Optionally, the space between the laterally spaced loops 1005 and/or the anterior extension 1008 are configured such that the opening of the cervix fits between the laterally separated elements. For example, there may not be lateral connecting elements on the cranial portion of the device. In some embodiments, this will inhibit strangulation of the cervix. Optionally, elements of the stent may be bent and/or undulating. For example, the wires of the retractors (e.g., extensions 1008, 1009 and/or loops1005 may undulate and/or be bent inward to provide more surface for support and/or incorporation into vaginal tissue). Optionally, anchors (for example a caudal portion of extensions 1008, 1009) may be bent outwards to make better contact with a supporting feature.

FIG. 11 is a perspective view of an undulating ring single filament stent 1100 in accordance with an embodiment of the current invention. In some embodiments, a stent 1100 may include an undulating filament (e.g., a wire and/or a polymer filament) bent to form a 3D ring. with multiple longitudinal (cranial/caudal) extensions 1108 joined on there cranial end by support members 1105 (e.g., arched and/or inwardly bent members). For example, the support members 1105 may support and/or retract the vaginal arch. Additionally or alternatively, the caudal ends of the extensions 1108 may be joined by connecting members 1102. In some embodiments, the support members 1108 may support the walls of vagina. In some embodiments, the connecting members 1102 and/or support members 1105 alternate such that if an organ were to prolapse into the center of the ring, it would not be strangled by a single uninterrupted ring. Optionally, some or all of the connecting members 1102 may be configured to rest on a feature of the vagina to anchor the stent 1100 inside the vagina. Alternatively or additionally, the stent 1100 may be anchored by intimate contact with the walls of the vagina. Optionally, a caudal portion of the support members 1108 and/or the connecting members 1102 may be bent outward.

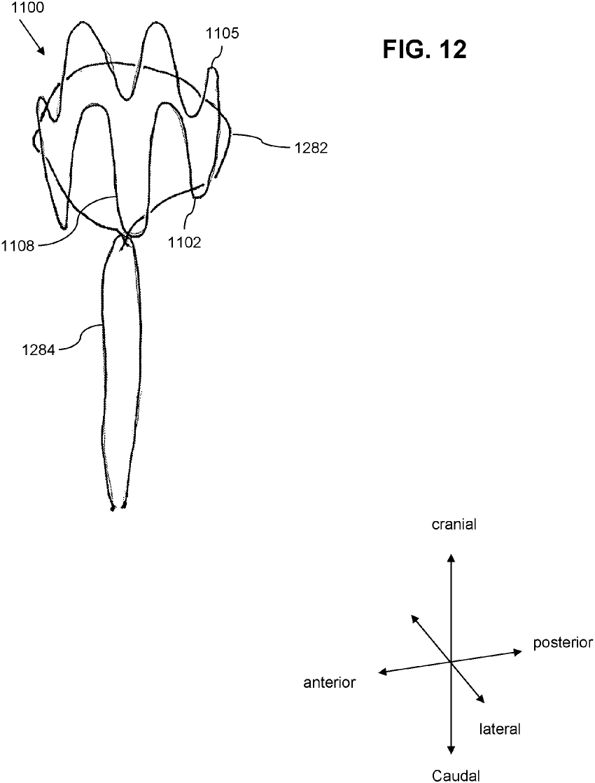

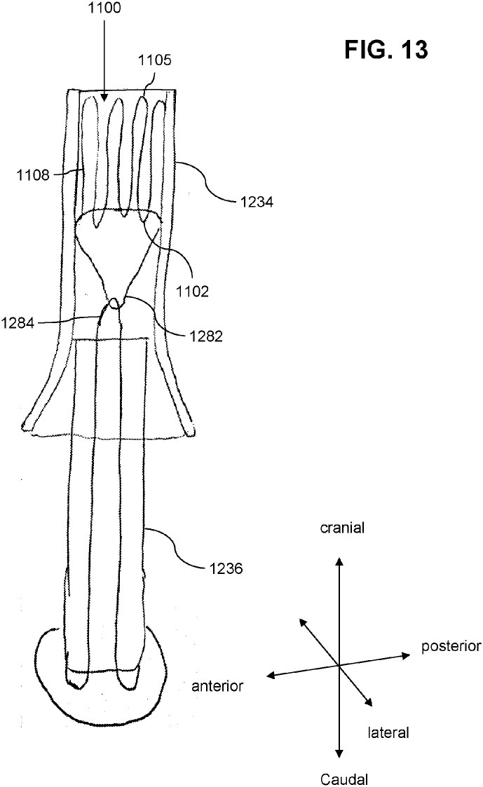

FIG. 12 is a schematic illustration of a stent 1100 being prepared for deployment in accordance with an embodiment of the current invention. In some embodiments, a stent 1100 may be prepared for delivery by connection to a collapsing element. Optionally, the collapsing element may be used to collapse the stent and/or to release the stent (e.g., allowing it to expand). For example, a cord 1182 (e.g., a string and/or wire) may be threaded through loops of the stent and/or wound around the stent. Optionally, when the cord 1282 is tightened, it may cause the stent 1100 to collapse from an expanded configuration (e.g., as illustrated in FIG. 12) to a collapsed configuration (e.g., as illustrated in FIG. 13).

In some embodiments, a release element may be supplied. For example, the release element may include a second cord 1284. For example, the release element may be used to control the stent 1100 during deployment and/or retrieval. For example, the stent 1100 may be held inside a deployment tube using the release element and/or at certain stages of deployment, the stent may be returned to a delivery tube using a release element. In some, embodiments, a release element includes a cord 1284 looped around a collapsing element (c.g., cord 1282) and/or a loop of the stent 1100.

In some embodiments, a release element and/or a collapsing element may be used with any of the embodiments of a stent and/or support device illustrated herein.

FIG. 13 illustrated a collapsed stent 1100 positioned inside a delivery tube 1236 in accordance with an embodiment of the current invention. For example, a delivery system may include a pusher 1234 and/or the stent 1100 may be collapsed and/or held to a pusher 1234 using a release cord 1284 and/or a collapsing cord 1282. For example, the pusher 1234 may be inserted into a proximal opening of the delivery tube 1236 and/or the collapsed stent may be held to a distal portion of the pusher 1234 using the release cord 1284 and/or collapse cored 1282. Optionally, a distal end of the release cord 1284 is available to a technician performing delivery of the device at the proximal portion of the delivery system. For example, the technician inserts the distal end of the delivery tube 1236 into the vagina of the patient and pushes out the cranial portion of the stent 1100 out the distal end of the tube 1236. Optionally, the pusher 1234 is used to push out the stent 1100. Optionally, the technician controls the delivery of and/or expansion of the device. For example, the technician may use release cord 1284 and/or the collapsing cord 1282 to release and/or the expand the stent 1100 gradually. For example, the device 1100 is pushed out of the delivery tube 1234 and/or the cranial portion starts to expand. Altnernatively or additionally, partial expansion may be limited by pulling taut on cord 1282 and/or expansion may be facilitated by releasing tension on cord 1282. In some embodiments, even after the device 1100 is fully pushed out of the tube 1234 expansion (of a cranial and/or caudal portion) may be limited by pulling taut on cord 1282 and/or expansion may be facilitated by releasing tension on cord 1282. Optionally, even after deployment and/or expansion, the device 1100 can be contracted by pulling cord 1282. For example, the device 1100 may be pulled back into the tube 1234 by cord 1282 and/or 1284. For example, pulling the device 1100 back into the tube 1234 may facilitate moving and/or removing the device 1100.

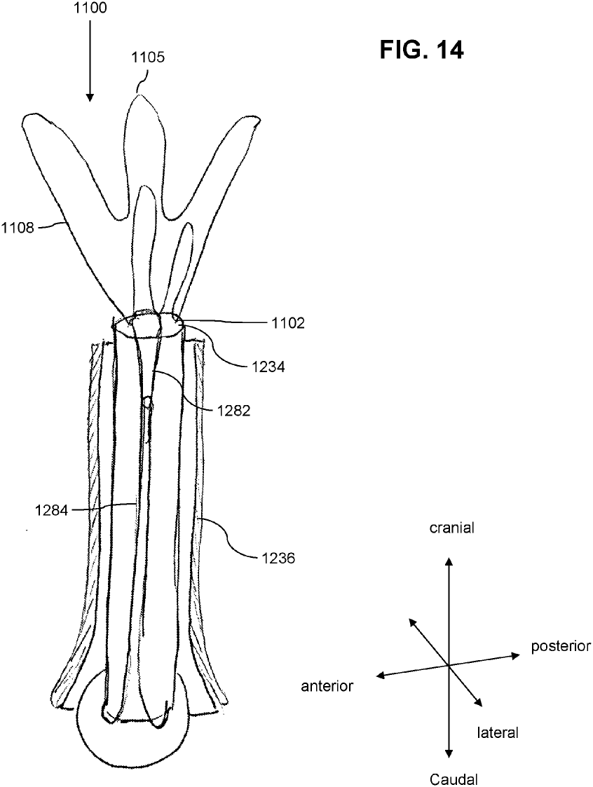

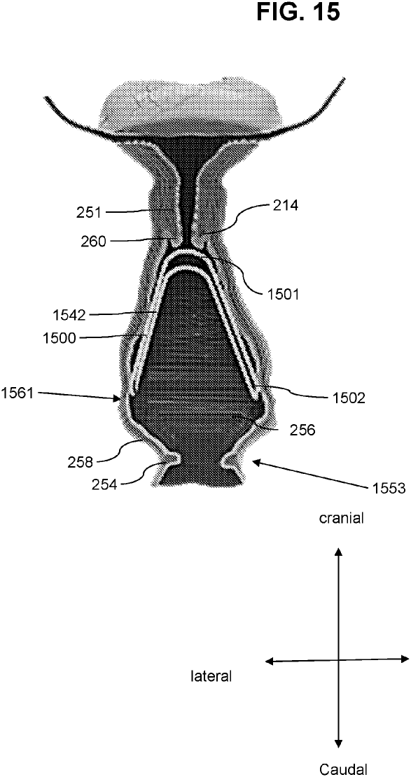

FIG. 14 is a schematic illustration of a stent in a partially deployed state in accordance with an embodiment of the current invention. In some embodiments, a stent 1100 is deployed gradually into a vagina. For example, the stent 1100 may be inserted into the vagina in a delivery tube 1236 and/or partially deployed. For example, an initial deployment may be limited to a pushing a cranial portion of the stent 1100 out of the tube 1236. Additionally or alternatively, the stent 1100 may be partially expanded. For example, tension on a release cord 1284 and/or an expansion cord 1282 may hold the stent in the partially deployed and/or partially expanded state. Optionally, while the device is partially deployed, a technician may check if it is positioned corrected in the vagina (for example, but pushing his finger around the delivery tube 1236 and feeling the position of the stent 1100 with respect to internal structures of the vagina. Optionally, when the stent 1100 is properly positioned, the release cord 1284 is separated from the stent 1100 and/or pulled out of the vagina leaving the stent 1100 in place and/or to expand fully. Optionally, the collapse cord 1282 remains on the stent 1100 and/or may be used later to collapse and/or move and/or remove the stent (for example, pulling the collapse cord 1282 may collapse the stent 1100while inside the vagina, making it easier to move and/or remove the stent 1100. Optionally when the stent expands in to the vagina, the filaments of the stent will become invaginated into the tissue (e.g., mucus tissue) of the walls of the vagina. Optionally, over the time vaginal tissue will grow over the filaments and/or incorporate the stent 1100 into the vaginal tissue.

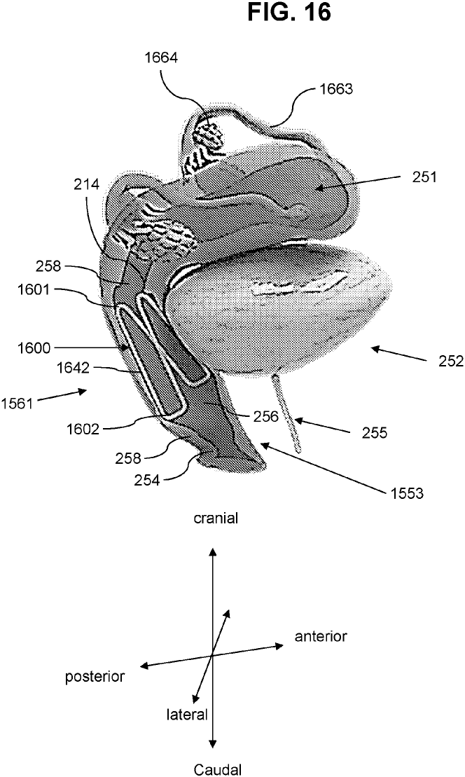

FIG. 15 is a is a schematic frontal view of a device 1500 including an installed in a vagina 1561 in accordance with an embodiment of the current invention. Various anatomical features are illustrated for example, a uterus 251, a pubic symphysis 254, a Vaginal epithelium 256, pelvic floor muscles and fascia 1553, Rectovaginal fascia 258, and a cervix 260. In some embodiments, the device 1500 includes a hollow elastic frame. Optionally, the frame is inserted into the vagina, in proper orientation with the cranial end 1501. For example, the cranial end 1501 may include an arch and/or support a vaginal vault 214. In some embodiments, lateral retractors 1542 spreading laterally in a caudal direction. Optionally, caudal end 1502 of the device (e.g., of the retractors 1542) rests on the pelvic floor muscles and facia 1553 for example on the rectovaginal fascia 258 and/or a pubic symphysis 254 and/or rectovaginal fascia. and the wire frame being in intimate contact with the vaginal walls and mucosa. In some embodiments, a device (for example stent 900 of FIG. 9) a device may include both lateral and/or anterior/posterior.

FIG. 16 is a is a schematic perspective view of female internal genitalia and a device 1600 including installed in a vagina 1561 in accordance with an embodiment of the current invention. Various anatomical features are illustrated for example, a uterus 251, a bladder 252, pelvic floor muscles and fascia 1553, a pubic symphysis 254, a urethra 255, Vaginal epithelium 256, Rectovaginal fascia 258, and a cervix 260. In some embodiments, the device 1600 includes a hollow elastic frame. Optionally, the frame is inserted into the vagina, in proper orientation. For example, the cranial end 1601 may include an arch and/or support a vaginal vault 214. Optionally, the frame is inserted into the vagina, in proper orientation with the cranial end 1601. For example, the cranial end 1601 may include an arch and/or support a vaginal vault 214. In some embodiments, lateral retractors 1542 spreading laterally in a caudal direction. Optionally, caudal end of the device (e.g., of the retractors 1642) rests on the pelvic floor. In some embodiments, a lateral caudal end 1602 of the device rests on the rectovaginal fascia 258 and/or a pubic symphysis 254. The wire frame is optionally in intimate contact with the vaginal walls and mucosa. In some cmbodiments, a device (for example stent 900 of FIG. 9) a device may include both lateral and/or anterior/posterior. It is of note that the devices of FIGS. 15 and 16 have an open cranial end 1501, 1601 leaving space for the cervix 260. Optionally there is no ring of in the device which must cause strangulation of a prolapsed organ (e.g., the cervix).

It is expected that during the life of a patent maturing from this application many relevant materials will be developed and the scope of the term filament is intended to include all such new technologies a priori.

As used herein the term “about” refers to ± 10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. When multiple ranges are listed for a single variable, a combination of the ranges is also included (for example the ranges from 1 to 2 and/or from 2 to 4 also includes the combined range from 1 to 4).

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An intravaginal device comprising:
a hollow elastic filament frame configured for intimate contact with vaginal walls;
a cranial end configured for supporting a vaginal dome and
a caudal end configured for resting on a pelvic floor for preventing dislodgment.

2. The device of claim 1, wherein said cranial end include members running in an anterior-posterior direction positioned to contact the vaginal dome on opposite lateral sides of a cervix.

3. The device of claim 2, wherein said caudal end includes one or more lateral running elements configured for resting on the pelvic floor and inhibiting dislodgment of the device.

4. The device of claim 1, wherein the filament has a diameter between ⅒ to 5 mm.

5. The device of claim 1, wherein the filament has a diameter between 3/10 to 3/2 mm.

6. The device of claim 1, wherein the caudal end is configured to rest on a perineal body.

7. The device of claim 1, wherein the caudal end is configured to rest on a pubic symphysis.

8. The device of claim 7, wherein the caudal end configured to rest on the pubic symphysis is provided with a notch to prevent pressure on a urethra.

9. The device of claim 1, wherein the frame delineates an axial passage for permitting unhindered sexual intercourse.

10. The device of claim 1, wherein the filament is made of a super-elastic material.

11. The device of claim 10 wherein said super-elastic material is selected from at least one of Nitinol and an elastic polymer.

12. The device of claim 1, wherein the frame is made of a nitinol wire.

13. The device of claim 1, wherein the frame is cut from a sheet.

14. The device of claim 1, wherein the frame is formed from a bent ring.

15. The device of claim 1, wherein the frame is saddle shaped with two caudal extensions.

16. The device of claim 1, wherein frame has a figure of eight shape including two loops each loop having a crossing point and two arched extensions connecting between the two crossing points.

17. The device of claim 1, wherein cranial end forms an arc.

18. The device of claim 1, wherein the cranial end of the device comprises two lateral wires, that are configured to support on opposite lateral sides of a uterine cervix.

19. The device of claim 1, wherein the frame is coated with a biocompatible material chosen from a group of: PTFE, POLYURETHANE, SILICONE.

20. A method for treating vaginal prolapse comprising:
inserting an open frame into a vagina;
placing the frame into intimate contact with vaginal walls and mucosa supporting a vaginal vault with a cranial end, of said open frame, and
resting a caudal end of the frame on a pelvic floor.

21. The method of claim 20 further comprising:
resting posterior caudal end of the frame on a perineal body.

22. The method of claim 20, further comprising:
incorporating said frame into vaginal walls.

23. The method of claim 20, further comprising:
compressing said frame before said inserting and
expanding said frame before said placing.

24. The method of claim 20, further comprising introducing said frame into a delivery tube prior to said inserting.

25. The method of claim 20, wherein said placing is done gradually.

26. The method of claim 25, wherein said placing is controlled using a collapsing element.

27. The method of claim 26, wherein said collapsing element includes a filament wrapped around the frame.

28. The method of claim 20, wherein an anterior portion of the caudal end of the device rests on the pubic symphysis.
